(19) United States
(12) Patent Application Publication
(10) Pub. No.: US 2004/0260134 A1
(43) Pub. Date: Dec. 23, 2004

(54) Process for producing cumene

(76) Inventors: Junpei Tsuji, Ichihara-shi (JP); Noriaki Oku, Ichihara-shi (JP)

Correspondence Address: SUGHRUE MION, PLLC, 2100 PENNSYLVANIA AVENUE, N.W., SUITE 800, WASHINGTON, DC, 20037, US

(21) Appl. No.: 10/489,184

(22) PCT Filed: Sep. 10, 2002
(86) PCT No.: PCT/JP02/09212

(30) Foreign Application Priority Data
Sep. 13, 2001 (JP) 2001-277701

(51) Int. Cl. C07C 1/20

(52) U.S. Cl. 585/469

(57) ABSTRACT

A process for producing cumene, which comprises subjecting cumyl alcohol to hydrogenolysis in the presence of a copper-based catalyst and using hydrogen having a carbon monoxide content of 5% by volume or less. The process is applied to cumene production in a hydrogenolysis step in propylene oxide production.

### TECHNICAL FIELD

The present invention relates to a process for producing cumene. More particularly, the present invention relates to a process for producing cumene, which includes subjecting cumyl alcohol to hydrogenolysis in the presence of a copper-based catalyst, and which has excellent characteristics that deterioration of an activity caused by reduction of a partial pressure of hydrogen and poisoning of the catalyst, can be prevented, whole volume of a reactor can be effectively utilized, and a purged amount of a gas can be suppressed to small when hydrogen is recycled.

### BACKGROUND ART

It is publicly known to produce cumene by hydrogenolysis of cumyl alcohol in the presence of a copper-based catalyst.

### DISCLOSURE OF THE INVENTION

The present inventors have studied regarding use of hydrogen which is used as a raw material for hydrogenolysis of cumyl alcohol, for example, hydrogen separated from a water gas obtained by reaction of methane and steam, and as a result, found that a small amount of carbon monoxide contained in hydrogen had a poisoning action on the catalyst. As a result of additional studies, they found that it was effective to suppress the amount of carbon monoxide to 5% by volume or less, and attained the present invention.

According to present invention, there is provided a process for producing cumene, which includes subjecting cumyl alcohol to hydrogenolysis in the presence of a copper-based catalyst, and which has excellent characteristics that deterioration of an activity caused by reduction of a partial pressure of hydrogen and poisoning of the catalyst, can be prevented, whole volume of a reactor can be effectively utilized, and a amount purged of a gas can be suppressed to small when hydrogen is recycled.

Namely, the present invention relates to a process for producing cumene, which comprises subjecting cumyl alcohol to hydrogenolysis in the presence of a copper-based catalyst, and using hydrogen containing 5% by volume or less of carbon monoxide.

### BEST MODE FOR CARRYING OUT THE INVENTION

The copper-based catalyst includes copper, Raney copper, copper-chromium, copper-zinc, copper-chromium-zinc, copper-silica, copper-alumina and compounds containing these.

The hydrogenolysis is usually carried out by contacting cumyl alcohol with hydrogen in the presence of the catalyst. The reaction can be carried out in a liquid phase using a solvent or in a gas phase. The solvent should be substantially inert to the reactants and the product. The solvent may be composed of a substance existing in a cumyl alcohol solution used. When, for example, cumyl alcohol is a mixture with cumene as the product, it is possible to use cumene as a solvent without adding a solvent, in particular. Other useful solvents include alkanes (e.g. octane, decane, dodecane) and aromatic monocyclic compounds (e.g. benzene, ethylbenzene, toluene) and the like. The hydrogenolysis temperature is usually 0 to 500° C. and preferably 30 to 400° C. In general, the pressure is advantageously 100 to 10000 kPa.

The hydrogenolysis can be advantageously carried out using the catalyst in the form of a slurry or a fixed-bed. The reaction can be carried out by a batch process, a semi-continuous process, or a continuous process.

In the present invention, it is necessary to suppress a content of carbon monoxide in a hydrogen gas to 5% by volume or less, in addition, it is preferable to use hydrogen in which total contents of carbon monoxide and carbon dioxide are suppressed to 15% by volume or less. Further, the content of carbon monoxide is preferably 3% by volume or less.

As a hydrogen resource, preferably, hydrogen obtained by using a water gas as a raw material, and separating and removing carbon monoxide therefrom so that the content of carbon monoxide becomes in the above-described range, is used.

The water gas is obtained by a catalytic reforming of a hydrocarbon such as methane or cokes with steam at high temperature, and is a mixed gas substantially composed of hydrogen, methane, carbon monoxide and carbon dioxide.

The activity deterioration of the catalyst in long time operation can be suppressed by satisfying the above-described condition. As a method for reducing carbon monoxide and carbon dioxide contained in hydrogen, for example, separation and removal methods such as absorbing removal by means of water, a solvent such as caustic water or an absorbent, adsorbing removal by means of a zeolite or a carbon molecular sieve, removal by means of reaction such as methanation, separation by means of a pressure swing absorbing method (PSA), and separation utilizing a semi-permeable membrane, are illustrated.

The process of the present invention can be conducted in hydrogenolysis step in production of propylene oxide comprising the following steps:

oxidation step: a step of obtaining cumene hydroperoxide by oxidizing cumene,

epoxidation step: a step of obtaining propylene oxide and cumyl alcohol by reacting cumene hydroperoxide obtained in the oxidation step with propylene in an excess amount in a liquid in the presence of a solid catalyst, and

hydrogenolysis step: a step of obtaining cumene by subjecting cumyl alcohol obtained in the epoxidation step to hydrogenolysis, and recycling the cumene to the oxidation step as a raw material for the oxidation step.

The oxidation step is a step for obtaining cumene hydroperoxide by oxidizing cumene. The oxidation of cumene is usually effected by autoxidation with oxygen-containing gas such as the air, an oxygen-enriched air or the like. The oxidation reaction may be carried out without any additive or with an additive such as an alkali. The reaction temperature is usually 50 to 200° C., and the reaction pressure is usually between the atmospheric pressure and 5 MPa.

In the oxidation using an additive, an alkali reagent used includes alkali metal compounds such as NaOH and KOH, alkaline earth metal compounds, alkali metal carbonates such as Na₂CO₃ and NaHCO₃, ammonia, (NH₄)₂CO₃, alkali metal ammonium carbonates and the like.

The epoxidation step is a step for obtaining propylene oxide and cumyl alcohol by reacting cumene hydroperoxide with propylene in an excess amount in a liquid phase in the presence of a solid catalyst for epoxidation. As the catalyst, from a viewpoint of obtaining the target product in high yield and under high selectivity, a solid catalyst containing a titanium-containing silicon oxide is preferable. The catalyst is preferably a so-called titanium-silica catalyst containing titanium chemically bound to silicon oxide. Examples thereof can include catalysts carrying a titanium compound on a silica carrier, catalysts in which a titanium compound is compounded with a silicon oxide by a co-precipitation or sol-gel method, titanium-containing zeolite compounds and the like.

Cumene hydroperoxide used as the raw material for the epoxidation step may be a dilute or thick purification or non-purification product.

The epoxidation is carried out by contacting propylene and cumene hydroperoxide with the catalyst. The reaction is conducted in a liquid phase using a solvent. The solvent should be a liquid under the reaction temperature and pressure, and substantially inert to the reactants and the product. The solvent may be composed of a substance existing in a solution of the hydroperoxide used. When, for example, cumene hydroperoxide is a mixture with cumene as the raw material, it is also possible to substitute cumene for a solvent, without adding a solvent in particular. Other useful solvents include aromatic monocyclic compounds (e.g. benzene, toluene, chlorobenzene, o-dichlorobenzene), alkanes (e.g. octane, decane, dodecane) and the like.

The epoxidation temperature is generally 0 to 200° C. and preferably 25 to 200° C. The pressure may be any pressure enough to keep liquid state of the reaction mixture. Generally, the pressure is advantageously 100 to 10,000 kPa.

The solid catalyst can be advantageously used in the form of a slurry or a fixed-bed. The fixed-bed is preferred in the case of a large-scale industrial operation. In addition, the reaction can be carried out by a batch process, a semi-continuous process, a continuous process or the like. When a liquid containing the raw materials for reaction is passed through a fixed-bed, the catalyst is not contained at all or substantially in a liquid mixture discharged from a reaction zone.

The molar ratio of propylene to cumene hydroperoxide supplied to the epoxidation step, is preferably 2/1 to 50/1. When the ratio is less than 2/1, the efficiency may be deteriorated because of decrease of the reaction rate. On the other hand, when the ratio is more than 50/1, there is a tendency that energy required in the recovery step becomes large because of increase of a propylene amount to be recycled.

The hydrogenolysis step is a step for obtaining cumene by subjecting cumyl alcohol obtained in the epoxidation step to hydrogenolysis, and the produced cumene is recycled to the oxidation step as a raw material of the above-described oxidation step, and is as mentioned above. In addition, in the present invention, it is preferable to set up a step of separation removal of carbon monoxide and further carbon dioxide before supplying hydrogen as a raw material to the hydrogenolysis step.

### EXAMPLE

The present invention is explained in detail by Examples, but is not limited thereto.

### Example 1

Into a fixed-bed flow reactor in which 100 g of a copper-silica catalyst was packed, a cumene solution containing 25% by weight of cumyl alcohol and hydrogen at rates of 2.7 g/minute and 1000 Ncc/minute, respectively were fed under conditions of 1.4 MPaG and 217° C. A reaction liquid obtained after 5 hours from the beginning of the feed, was analyzed, and the decomposition activity of cumyl alcohol was shown in Table 1.

### Example 2

A reaction was carried out in the same manner as in Example 1 except that hydrogen containing carbon monoxide of 1% by volume was fed into a fixed-bed reactor. The result was shown in Table 1.

### Comparative Example 1

A reaction was carried out in the same manner as in Example 1 except that hydrogen containing carbon monoxide of 10% by volume was fed into a fixed-bed reactor. The result was shown in Table 1.

TABLE 1

| | Example | Example | Comparative |
|---|---|---|---|
| | 1 | 2 | Example 1 |
| Carbon monoxide | 0 | 1 | 10 |
| concentration | | | |
| (% by volume) | | | |
| Cumyl alcohol | 9.3 | 9.3 | 8.3 |
| decomposition | | | |
| activity (k) | | | |

### INDUSTRIAL APPLICABILITY

As described above, according to the present invention, a process for producing cumene, which comprises subjecting cumyl alcohol to hydrogenolysis in the presence of a copper-based catalyst, and which has excellent characteristics that deterioration of an activity caused by reduction of a partial pressure of hydrogen and poisoning of the catalyst, can be prevented, whole volume of a reactor can be effectively utilized, and the amount purged of a gas can be suppressed to small when hydrogen is recycled, can be provided.

1. A process for producing cumene, which comprises subjecting cumyl alcohol to hydrogenolysis in the presence of a copper-based catalyst, and using hydrogen containing 5% by volume or less of carbon monoxide.

2. The process according to claim 1, wherein the total amount of carbon monoxide and carbon dioxide is 15% volume or less.

3. A process for propylene oxide, which comprises the following steps:
oxidation step: a step of obtaining cumene hydroperoxide by oxidizing cumene;
epoxidation step: a step of obtaining propylene oxide and cumyl alcohol by reacting cumene hydroperoxide obtained in the oxidation step with propylene in an excess amount in a liquid in the presence of a solid catalyst; and
hydrogenolysis step: a step of obtain cumene by subjecting cumyl alcohol obtained in the epoxidation step to hydrogenolysis, and recycling the cumene to the oxidation step as a raw material for the oxidation step,
wherein the production of cumene is carried out by the process of claim 1.

4. The process according to claim 3, wherein a step of separation-removal for reducing the carbon monoxide content to 5% by volume or less before supplying hydrogen as a raw material to the hydrogenolysis step, is added.

5. The process according to claim 3, wherein a step of separation-removal for reducing the carbon monoxide content to 5% by volume or less and the total contents of carbon monoxide and carbon dioxide to 15% by volume or less before supplying hydrogen as a raw material to the hydrogenolysis step is added.

6. The process according to any one of claims 1 to 5, wherein, as a hydrogen source, hydrogen obtained by separating and removing carbon monoxide from a water gas to reduce the content thereof to 5% by volume or less, is used.
